(19) United States
(12) Patent Application Publication
(10) Pub. No.: US 2023/0201395 A1
(43) Pub. Date: Jun. 29, 2023

(54) SYSTEM AND METHOD FOR SANITIZING AT LEAST ONE SURFACE

(71) Applicant: SAFE ANTIVIRUS TECHNOLOGIES INC., Toronto (CA)

(72) Inventors: Steven Christopher MARTIN, Toronto (CA); Kieran Alexander Singh MARTIN, Toronto (CA); Jamie A. GONZALEZ, Toronto (CA); Kevin Hubert Menzies ANDRADE, Toronto (CA); Liann DSOUZA, Scarborough (CA); Tony DI PIETRO, Brampton (CA)

(21) Appl. No.: 17/996,316

(22) PCT Filed: Apr. 15, 2021
(86) PCT No.: PCT/CA2021/050508

§ 371 Date: Oct. 14, 2022

Related U.S. Application Data
(60) Provisional application No. 63/010,614, filed on Apr. 15, 2020.
(60) Provisional application No. 63/021,456, filed on May 7, 2020.
(60) Provisional application No. 63/074,898, filed on Sep. 4, 2020.

(51) Int. Cl. A61L 2/24; A61L 2/28; A61L 2/10

(52) U.S. Cl. CPC A61L 2/24; A61L 2/28; A61L 2/10; A61L 2202/14; A61L 2202/11

(57) ABSTRACT

A method and system for sanitizing at least one surface in an area to be sanitized are provided. At least one illumination module is positioned to radiate the at least one surface with sanitizing electromagnetic radiation (SER). A dosimeter is positioned to measure a level of SER emitted by the at least one illumination module or received by the at least one surface. A control module is in communication with the dosimeter to receive the level of SER emitted by the at least one illumination module or received by the at least one surface, determine an appropriate level of SER to be provided to the at least one surface at least partially based on the level of SER emitted or received by the at least one surface, and control the at least one illumination module to radiate the at least one surface with the determined appropriate level of SER.

### CROSS-REFERENCE TO RELATED APPLICATIONS

This application claims the benefit of U.S. Provisional Application No. 63/010,614 filed Apr. 15, 2020, U.S. Provisional Application No. 63/021,456 filed May 7, 2020, and U.S. Provisional Application No. 63/074,898 filed Sep. 4, 2020, the contents of all of which are incorporated herein by reference in their entirety.

### FIELD

The specification relates generally to sanitization, and in particular to a system and method for sanitizing at least one surface.

### BACKGROUND OF THE DISCLOSURE

Ensuring appropriate levels of sanitization as means to limit contamination, pathogen transmission, and disease has proven to be a considerable challenge. This has become even more evident as the COVID-19 moved from a localized outbreak to a global pandemic.

It is instructive to examine how the COVID-19 illness spread as a fresh proxy for the overall issue, and to help determine what could have been done to limit this spread. COVID-19, as with many viruses and communicable illnesses, transmits primarily through person-to-person exchange, both by proximity, and from contaminated objects. With people in close proximity to each other droplets from sneezing and coughing can carry the virus from person to person, and commonly touched surfaces if contaminated with virus-containing droplets can similarly cause transmission. In particular, long after the issue of airborne transmission has passed, contaminated surfaces remain a significant mode of transmission. Touching a contaminated doorknob, for example, carries the risk for anyone touching it of transferring the virus to their hands, which could lead ultimately to contracting the illness if they touch their nose, mouth, or face. In order to reduce transmission, steps must be taken to reduce the amount of airborne and surface live virus that is available for transmission. Frequent hand washing and surface wipe-downs help, but they depend on an individual's vigilance to be effective.

The issue of enhanced sanitization has been elevated in our collective consciousness since the onset of the global COVID-19 pandemic, however there are additional uses for the technology than can greatly enhance the levels of sanitization in our daily lives. For example, in manufacturing systems for food and related consumables there are requirements that have been set for by organizations such as NSF, the USFDA, and Health Canada that specify the need to ensure an appropriate level of cleanliness is achieved in all systems that touch food, medicines, and other items intended for human consumption.

### SUMMARY OF THE DISCLOSURE

In one aspect, there is provided a system for sanitizing at least one surface in an area to be sanitized, comprising: at least one illumination module positioned to radiate the at least one surface with sanitizing electromagnetic radiation (SER); a dosimeter positioned to measure a level of SER emitted by the at least one illumination module or received by the at least one surface; and a control module in communication with the dosimeter to receive the level of SER emitted by the at least one illumination module or received by the at least one surface, determine an appropriate level of SER to be provided to the at least one surface at least partially based on the level of SER emitted by the at least one illumination module or received by the at least one surface, and control the at least one illumination module to radiate the at least one surface with the determined appropriate level of SER.

The system can further include a use sensor positioned to determine a level of use of the area to be sanitized, and the control system determines the appropriate level of SER to be provided to the at least one surface at least partially based on the level of use of the area to be sanitized. The use sensor can be a motion sensor. The at least one surface can include a surface of a handrail or a conveyor belt.

The control module can include storage storing a set of rules specifying conditions during which SER can be radiated by the at least one illumination module, and the control module can control the at least one illumination module to only emit SER when the set of rules are satisfied.

At least one of the set of rules can specify the absence of occupation or movement in the area to be sanitized.

At least one of the set of rules can specify the closure or a lock state of a door or port providing access to the area to be sanitized.

At least one of the set of rules can specify a deactivation of standard visible illumination in the area to be sanitized.

The control module can generate one of an audible alert and a visual alert when at least one of the set of rules is unsatisfied.

The control module can log a failure event in a database in the storage when at least one of the set of rules is unsatisfied.

The control module can operate the illumination module to produce the appropriate level of SER according to at least one sanitization profile that specifies a duration, an intensity, and at least one wavelength of light. The control module can perform a calibration to verify the appropriate level of SER by directing the at least one illumination module to generate a level of SER, measuring the intensity of SER using the dosimeter, and registering an adjustment for the at least one sanitization profile based on the measured intensity of SER. The calibration can be performed during the provisioning of SER by the at least one illumination module to update the intensity in real-time.

The control module can be in communication with a server to receive one or more updated sanitization profiles from the server.

The at least one illumination module can be equipped to produce at least two wavelengths.

The at least one illumination module can be equipped to produce at least two wavelengths of SER.

The system can further include a surface contamination sensor positioned to measure a level of surface contamination on the at least one surface, wherein the control module is in communication with the surface contamination sensor to receive the level of surface contamination and generate an alert when the level of surface contamination on the at least one surface surpasses a threshold.

The control module can provide at least one of an audible alert and a visual alert prior to controlling the at least one illumination module to radiate the at least one surface.

In another aspect, there is provided a system for sanitizing at least one surface of an area to be sanitized, comprising: at least one illumination module positioned to radiate the at least one surface with sanitizing electromagnetic radiation (SER); a use sensor positioned to determine a level of use of the area to be sanitized; and a control system in communication with the use sensor to receive the level of use of the area to be sanitized, determine an appropriate level of SER to be provided to the at least one surface at least partially based on the level of use, and control the at least one illumination module to radiate the at least one surface with the determined appropriate level of SER.

The at least one surface can include a moving surface, and the use sensor can detect movement of the moving surface and determine the appropriate level of SER to be provided to the at least one surface at least partially based on the movement of the moving surface. The use sensor can detect a rate of the movement of the moving surface and determine the appropriate level of SER to be provided to the at least one surface at least partially based on the rate of movement of the moving surface.

In a further aspect, there is provided a method for sanitizing at least one surface in an area to be sanitized, comprising: measuring a level of sanitizing electromagnetic radiation (SER) emitted by at least one illumination module or received by the at least one surface; determining an appropriate level of SER to be provided to the at least one surface at least partially based on the level of SER emitted by the at least one illumination module or received by the at least one surface; and controlling the at least one illumination module to radiate the at least one surface with the determined appropriate level of SER.

The method can further comprise: determining, via a use sensor, a level of use of the area to be sanitized; and determining, via a control module, the appropriate level of SER to be provided to the at least one surface at least partially based on the level of use of the area to be sanitized.

The use sensor can be a motion sensor.

The at least one surface can include a surface of a handrail or a conveyor belt.

The method can further comprise controlling, via a control module, the illumination module to only emit SER when a set of rules stored in storage are satisfied, the set of rules specifying conditions during which SER can be radiated by the at least one illumination module.

At least one of the set of rules can specify the absence of occupation or movement in the area to be sanitized.

At least one of the set of rules can specify the closure or a lock state of a door or port providing access to the area to be sanitized.

At least one of the set of rules can specify a deactivation of standard visible illumination in the area to be sanitized.

The method can further comprise generating, via the control module, one of an audible alert and a visual alert when at least one of the set of rules is unsatisfied.

The method can further comprise logging, via the control module, a failure event in a database in the storage when at least one of the set of rules is unsatisfied.

The method can further comprise controlling, via the control module, the at least one illumination module to produce the appropriate level of SER according to at least one sanitization profile that specifies a duration, an intensity, and at least one wavelength of light.

The method can further comprise performing, via the control module, a calibration to verify the appropriate level of SER by directing the at least one illumination module to generate a level of SER, measuring the intensity of SER using the dosimeter, and registering an adjustment for the at least one sanitization profile based on the measured intensity of SER.

The calibration can be performed during the provisioning of SER by the at least one illumination module to update the intensity in real-time.

The method can further comprise receiving, via the control module, one or more updated sanitization profiles from a server with which the control module is in communication.

The at least one illumination module can be equipped to produce at least two wavelengths.

The at least one illumination module can be equipped to produce at least two wavelengths of SER.

The method can further comprise: measuring, via a surface contamination sensor, a level of surface contamination on the at least one surface; receiving, via the control module, the level of surface contamination from the surface contamination sensor; and generating an alert when the level of surface contamination on the at least one surface surpasses a threshold.

The method can further comprise providing, via the control module, at least one of an audible alert and a visual alert prior to controlling the at least one illumination module to radiate the at least one surface.

In yet another aspect, there is provided a method for sanitizing at least one surface in an area to be sanitized, comprising: determining, via a use sensor, a level of use of the area to be sanitized; determining, via a control module, an appropriate level of sanitizing electromagnetic radiation (SER) to be provided by at least one illumination module to the at least one surface at least partially based on the level of use; and controlling the at least one illumination module to radiate the at least one surface with the determined appropriate level of SER.

The at least one surface can include a moving surface, and the method can further comprise: detecting, via the use sensor, movement of the moving surface; and determining, via a control module, the appropriate level of SER to be provided to the at least one surface at least partially based on the movement of the moving surface.

The method can further comprise: detecting, via the use sensor, a rate of the movement of the moving surface; and determining the appropriate level of SER to be provided to the at least one surface at least partially based on the rate of movement of the moving surface.

Other technical advantages may become readily apparent to one of ordinary skill in the art after review of the following figures and description.

### BRIEF DESCRIPTIONS OF THE DRAWINGS

For a better understanding of the embodiment(s) described herein and to show more clearly how the embodiment(s) may be carried into effect, reference will now be made, by way of example only, to the accompanying drawings in which:

FIG. 1 is a schematic diagram of various components of a sanitizing electromagnetic radiation (SER) system for sanitizing one or more surfaces in accordance with an embodiment;

FIG. 2 is a schematic diagram of various physical and logical components of a control module of the SER system of FIG. 1;

FIG. 3 is a flowchart of the general method of sanitizing surfaces using the SER system of FIGS. 1 and 2;

FIG. 4 is a flowchart of the general method of initialization of the SER system of FIGS. 1 and 2;

FIG. 5 is a flowchart of the general method of calibration of the SER system of FIGS. 1 to 2;

FIG. 6 is a flowchart of the general method of sanitization of a surface using the SER system of FIGS. 1 to 2;

FIG. 7. is a flowchart of the general method of obtaining feedback by the SER system of FIGS. 1 and 2;

FIG. 8A is a schematic view of a buy online, pickup in store (“BOPIS”) locker in accordance with another embodiment;

FIG. 8B shows the BOPIS locker of FIG. 8A with the door thereof opened to show an internal compartment in which products are positioned;

FIG. 9 is a flowchart of a sanitization method of the BOPIS locker of FIGS. 8A and 8B;

FIG. 10 is a flowchart of an alternative sanitization method of the BOPIS locker of FIGS. 8A and 8B;

FIG. 11A is a top, front, right-side view of an escalator handrail sanitizer shown positioned over a section of escalator handrail in accordance with a further embodiment;

FIG. 11B is a top, front, right-side view of the escalator handrail sanitizer of FIG. 11A positioned over the section of escalator handrail after removal of two component covers;

FIG. 11C is a bottom, front, right-side view of the escalator handrail sanitizer of FIG. 1B;

FIG. 12 is a flowchart of the control system for the escalator handrail sanitizer of FIGS. 11A to 11C;

FIG. 13 is schematic view of an SER system for sanitizing transit user-facing surfaces in accordance with yet another embodiment;

FIG. 14 is an exemplary ceiling-mounted transit system occupancy sensor/dosimeter for use with the SER system for sanitizing transit user-facing surfaces of FIG. 13; and

FIG. 15 is a schematic view of an SER system for sanitizing a surface of a conveyor belt.

Unless otherwise specifically noted, articles depicted in the drawings are not necessarily drawn to scale.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiment or embodiments described herein. However, it will be understood by those of ordinary skill in the art that the embodiments described herein may be practiced without these specific details. In other instances, well-known methods, procedures and components have not been described in detail so as not to obscure the embodiments described herein. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description. It will also be noted that the use of the term “a” or “an” will be understood to denote “at least one” in all instances unless explicitly stated otherwise or unless it would be understood to be obvious that it must mean “one”.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

Exposure to electromagnetic radiation, including ultraviolet (UV) light and blue light is a well-understood sanitization strategy and can achieve rapid inactivation of viruses and other pathogens upon direct illumination. Other wavelengths of light may also provide a sanitization effect. Any such electromagnetic radiation that is capable of enhancing sanitization and/or inactivating or reducing viruses and other pathogens or contamination shall collectively be referred to herein as sanitizing electromagnetic radiation (SER). Sanitizing electromagnetic radiation can include UV light, blue light, and/or other wavelengths of light.

SER lighting can provide a significant and cost-effective enhancement of all sanitization protocols, and when included with appropriate controls in a monitored system, they can be safe, autonomous, and not dependent on the time or vigilance of healthcare, cleaning, and maintenance workers to achieve their efficacy.

In various embodiments disclosed herein, an appropriate level of SER that has been calibrated to reduce pathogen, contamination, and other deleterious biological elements (“viral load”) through the use of integrated feedback control is provided to sanitize surfaces.

By providing a control system to determine how much SER is appropriate and then controlling an SER system of one or more illumination modules to deliver the appropriate level of SER, the level of SER can be customized to accommodate dynamically changing needs. The control system can measure at least one of a level of surface contamination on one or more surfaces to be sanitized, a level of estimated exposure of the surface(s) to contaminants based on use of a system of which the surface(s) form(s) a part of, and/or a level of SER received by the surface(s). Further, the control system can determine an appropriate level of SER to be applied to the surface(s) using the level of surface contamination on the surface(s), the level of estimated exposure of the surface(s) to contaminants based on use of a system of which the surface(s) form(s) a part of, and/or the level of SER received by the surface(s). The control system can then control the one or more illumination modules to radiate the surface(s) at the determined appropriate level.

In some embodiments, the control system can include a dose meter, or dosimeter, that reads the amount of SER that impinges on the surface(s) from the SER system, the level of assurance of operation is greatly enhanced. This is akin to having a speedometer in a vehicle to provide a “guarantee” of the speed of the vehicle, allowing the operator greater control, and providing a means to achieve compliance with regulations (for example, a speed limit). In the absence of such a system, the vehicle will still function, but it is operated with no feedback beyond the operator's ability to judge “fast” versus “faster”.

The core of the technology is the close integration of the sanitizing system and the control loop to adjust the AVLS light delivered so that desired amounts of light can be reliably delivered upon each operation.

The method of achieving this calibration, that is the feedback control system, comprises a means to measure the light delivered to a surface, space, or area, and a means of adjusting the SER so that an appropriate dose is achieved. The feedback can be real-time closed loop, a one-time closed loop calibration, or even a one-time calibration set-up. Disclosed herein is a framework for rapid deployment of a robust illumination module, with appropriate feedback control, that can be integrated with required room illumination, if desired, and installed in virtually any user-facing area, such as a high touch, high traffic areas, to achieve appropriate and/or greater levels of sanitization.

This solution can be to be installed in offices, boardrooms, public washrooms, on escalator handrails, in elevators, on conveyor systems such as those used at supermarket checkouts, and other high-traffic areas that would help minimize the spread of viral load, and thus reduce the spread of pathogens.

In one embodiment this lighting system can also supply standard “white-light” room lighting, and, in addition to the closed-loop feedback, can monitor occupancy as both a safety interlock and to ensure that spaces are appropriately exposed to SER illumination once vacated. Moreover, in some embodiments the system includes wired or wireless connectivity to dosimetry and data-logging, for quality assurance, and subsequent data-analytics allowing tracking of effectiveness and additional safety protocols.

In some embodiments the SER system can be deployed in food and pharma to enhance the existing clean-in-place protocols, allowing for enhanced assurance and reduced contamination.

The disclosed SER systems can provide enhanced sanitization of viruses and other pathogens in all environments in which it is used, increasing peace of mind for occupants, and greatly improved health and safety beyond the current COVID-19 crisis.

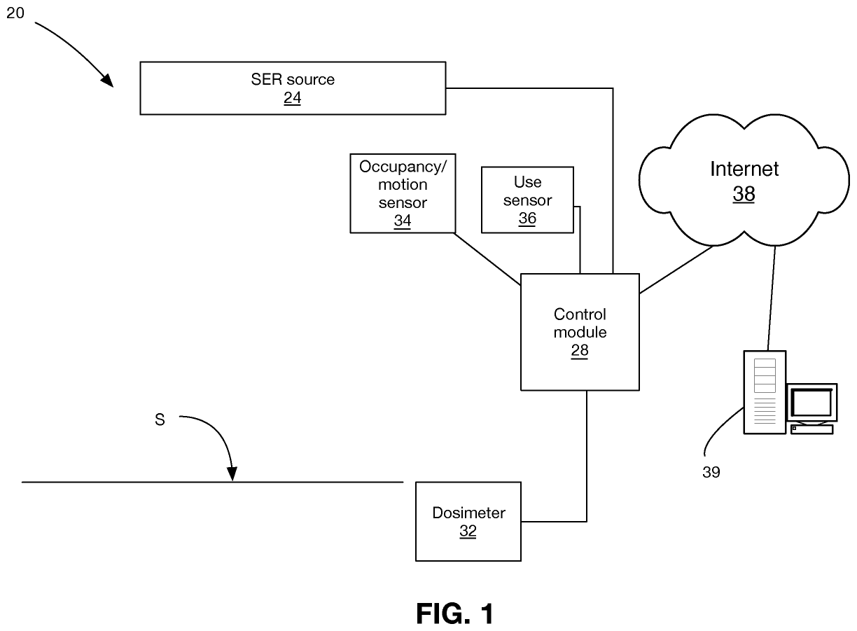

FIG. 1 is a schematic diagram of various components of an SER system 20 for sanitizing a surface or surfaces of an area to be sanitized in accordance with an embodiment. The SER system 20 includes an illumination module 24 that is, in a present embodiment, LED-based, but can be constructed in any suitable manner known to those skilled in the art. In preferred embodiments, the illumination module 24 includes illumination sources that can produce SER of differing wavelengths selected as effective sanitizers that can be used alone or in tandem, and also includes a standard white light illumination source. A control module 28 is wired or wirelessly in communication with the illumination module 24 to control the intensity, duration, timing, and/or frequency of the SER generated by the illumination module 24. The control module 28 is also or wirelessly in communication with a dosimeter 32, an occupancy/motion sensor 34, and a use sensor 36. The dosimeter 32 can measure the intensity, duration, timing, and wavelength(s) of SER that is expected that a surface or surfaces to be sanitized receive(s). In addition, the dosimeter 32 can be used to measure the intensity of standard visible light in an area to be sanitized. Preferably, the dosimeter is positioned adjacent to a surface S to be sanitized by the SER system 20. The occupancy/motion sensor 34 can detect the presence of a person in the area to be sanitized, and can be any suitable type of sensor for this purpose, such as proximity sensors, image sensors, infrared beam obstruction sensors, etc. The use sensor 36 can determine the timing and level of use that the area to be sanitized or a part thereof receives, and can be any suitable type of sensor, such as an image sensor, a motion sensor, etc. The control module 28 is connected to a communications network such as the Internet 38 and is in communication with a server 39 that manages an external database of sanitization profiles detailing regimens of doses of SER illumination, as well as calibration data.

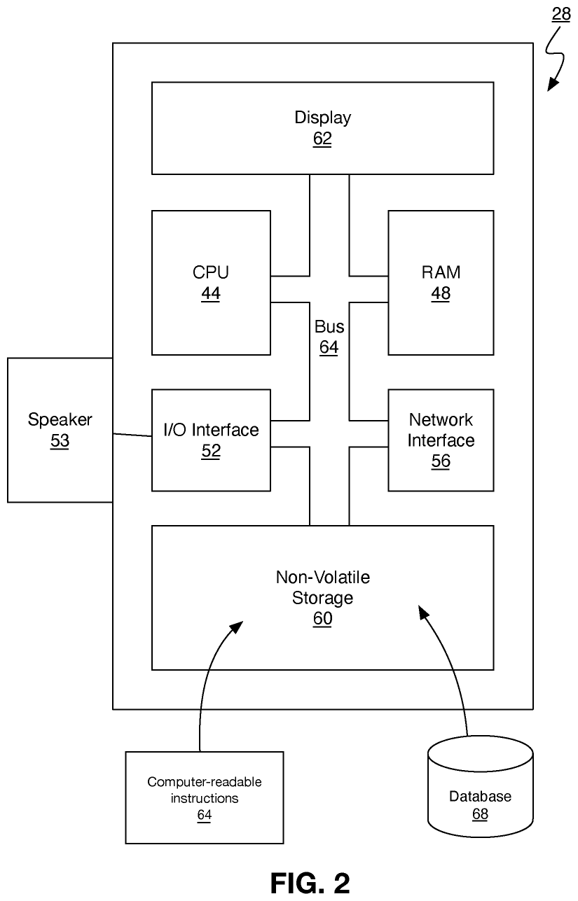

Now with reference to FIGS. 1 and 2 is shown various physical and logical components of the control module 28. The control module 28 includes a central processing unit (“CPU”) 44, random access memory (“RAM”) 48, an input/output (“I/O”) interface 52, a network interface 56, non-volatile storage 60, a display 62, and a local bus 64 enabling the CPU 44 to communicate with the other components. The CPU 44 executes at least an operating system, and a SER control application. RAM 48 provides relatively responsive volatile storage to the CPU 44. The I/O interface 52 allows for input to be received from one or more devices, such as buttons on the control module 28, a keyboard, a mouse, etc., and outputs information to output devices, such as a speaker 53 connected to the I/O interface 52 to generate an audible alert, an external display, etc. The I/O interface 52 can also be in connected to the illumination module 24, the dosimeter 32, the occupancy/motion sensor 34, and the use sensor 36. The network interface 56 permits communication with other computing devices over wired and/or wireless computer networks such as Internet. The non-volatile storage 60 stores the operating system, programs including computer-executable instructions 64 for implementing the SER control application, and a database 68 of configuration data for configuring the SER system 20, and log data captured by the control module 28. During operation of the control module 28, the operating system, the computer-readable instructions 64, the configuration data, and the log data may be retrieved from non-volatile storage 60 and placed in RAM 48 to facilitate execution. The display 62 enables messages or status to be presented to a user.

Returning again to FIG. 1, the illumination spectrum of both the standard room “white” light and the SER lights produced by the illumination module 24 may be selected to meet requirements of a given application. The white light may be selected to meet the illumination needs of the user, ranging from approximately 2,500K to 6,000K (warm white to cool/blue white).

The spectrum of the SER lights can be selected, and may in some embodiments be adjustable in real-time, from approximately 200 nm in the UVC range to over 400 nm in the UVA range, although in some embodiments shorter or longer wavelengths may be preferred. The selection is based upon the particular sanitization requirements, the required duration of sanitization, other operating conditions (including room configuration), and safety.

The wavelength used for sanitization may be altered throughout a sanitization cycle, to improve sanitization generally, to improve selective sanitization and elimination of specific pathogens, to improve the time required for a sanitization operation, or for other reasons. Wavelengths may be skewed during an exposure cycle, combined, added, subtracted, or applied in other manners to affect a sanitization cycle. Such a wavelength profile, encompassing the wavelengths used, the skewing, if any, the additive or subtractive nature of wavelengths used, and other alterations to the sanitization wavelength can be employed.

Wavelength profiles can include linear skewing, exponential skewing, logarithmic skewing, or more complex combinations of these and other profiles. Wavelength profiles may also include periods with multiple wavelengths, varying in intensity with intensity profiles, as described below. Wavelength profiles may be determined empirically using the collected data from the operation of the SER system 20, through literature searches, through a combination of both empirical and theoretical, or through other means. Wavelength profiles and may or may not operate in concert with either of intensity or duration profiles.

Specified wavelength profiles can be transmitted to the illumination module 24, via initiation by either the illumination module 24 or the server 39, and a means to track the profile used in each sanitization cycle can be provided, such as local storage means within the illumination module or the control module 28.

Intensity may be pre-set or may be adjustable to allow for consideration of sanitization requirements, operational limitations or needs, exposure times, and safety. There may be communication between the illumination module 24 and the dosimeter 32 to provide calibration and/or feedback information for the correct illumination operation for the desired sanitization protocol. Adjustments may be made between sanitization cycles in an open loop mode to account for degradation of the SER light which may be due to dust/dirt on illumination lenses or reflective surfaces, reduction in efficiency of the LED emitter devices of the SER illumination module 24 over time, or for other reasons. Intensity may also be adjusted in real-time through feedback from the wired or wireless dosimeter 32, which can include PID control, bang-bang control, or other feedback algorithms. Intensity may be selected to ramp up or down over a sanitization cycle, or to adopt other more complex profiles either during, or between cycles, for greater efficacy, or other reasons. A profile may be selected from a database of illumination profiles to effect sanitization preferentially for particular pathogens, to achieve a higher degree of sanitization, or for other reasons.

Intensity profiles may be linear increasing or decreasing, exponential increasing or decreasing, logarithmic increasing or decreasing, or more complex combinations of these and other profiles. Illumination profiles may also include periods with SER generation halted, or periods with one or more bright flashes of SER. Intensity profiles may or may not operate in concert with either of wavelength or duration profiles. In one embodiment, the SER illumination emitted by the illuminator module 24 may be ramped slowly from the start of the cycle for increased safety.

Profiles may be determined empirically using the collected data from operation of the SER system 20, through literature searches, through a combination of both empirical and theoretical, or through other means.

Specified intensity profiles can be transmitted to the illuminator module 24, via initiation by either the illuminator module 24, the control module 28, or the server 39, and a means to track the profile used in each sanitization cycle can be provided, such as local storage means within the illuminator module 24 or the storage 60 of the control module 28.

The duration of the sanitization cycle may be pre-set or may be adjustable to allow for consideration of sanitization requirements, operational limitations or needs, exposure times, and safety. There may be communication between the wired or wireless sensors to provide calibration and/or feedback information for the correct illumination operation for the desired sanitization protocol. Adjustments, such as extensions of the exposure time, or reductions to the duration of exposure, may be made between sanitization cycles in an open loop mode to account for degradation of SER light which can arise due to dust/dirt on illumination lenses or reflective surfaces, reduction in efficiency of the LED emitter devices of the illumination module 24 over time, or for other reasons.

Duration may also be adjusted in response to feedback from the wired or wireless dosimeter 32, which can include PID control, bang-bang control, or other feedback algorithms. Duration may be selected to increase or decrease during a sanitization cycle, to increase or decrease for the next sanitization cycle, or to change in other ways for greater efficacy, to meet sanitization requirements, or other reasons. A duration profile may be selected from a set of duration profiles in the database 68 to effect sanitization preferentially for particular pathogens, to achieve a higher degree of sanitization, or for other reasons.

Duration profiles may include periods with the SER illuminator elements of the illumination module 24 off, or periods with one or more bright flashes of SER, and may or may not operation in concert with either of intensity or wavelength profiles. Duration profiles may be determined empirically using the collected data from the operation of the SER system 20, through literature searches, through a combination of both empirical and theoretical, or through other means.

The wireless or wired dosimeter 32 allows factory, jobsite, transit vehicle, or other, measurement of the SER light dose emitted from the illumination module, which may be used for calibration, real-time monitoring, data collection, assurance of the delivered SER dose, or for other reasons. In some embodiments, the dosimeter 32, the occupancy/motion sensor 34, and the use sensor 36 can be integrated into a single physical device, and can include other sensors or monitoring systems. Where the dosimeter 32, the occupancy/motion sensor 34, and the use sensor 36 are exposed to contact, such as by humans, the dosimeter 32, the occupancy/motion sensor 34, and the use sensor 36 can be ruggedized, made tamper-proof to prevent unauthorized alteration, or otherwise shielded or protected to prevent compromise of the dosimeter 32, the occupancy/motion sensor 34, and the use sensor 36.

The dosimeter 32 has one or more sensors capable of detecting the SER light emitted from the illumination module 24 for sanitization, and a means to record, display, transmit, or otherwise utilize the dose information collected. The dose information may be used as part of the control system for the sanitization cycle, including providing feedback for adjustments of either the intensity, the wavelength, or the duration of the cycle to achieve the required dose. The dosimeter 32 may also be used to allow monitoring, and control of any or all of wavelength profiles, intensity profiles, and duration profiles. The dosimeter may contain internal memory to record sanitization and other events, may be cloud connected to allow remote monitoring and historian recording of sanitization and other events, or may have other means or recording and communicating sensor readings. The dosimeter 32 may include connectivity to external controls, including mobile device applications, IoT devices, or other control or data collection devices.

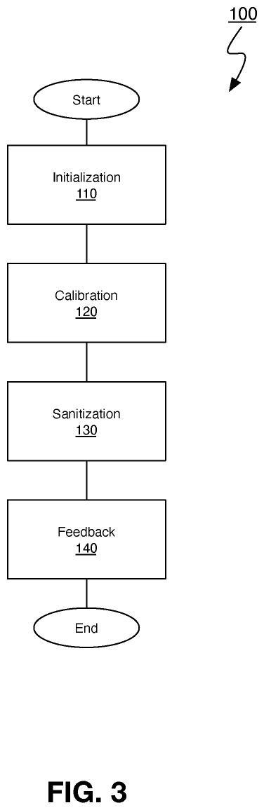

FIG. 3 shows the general method 100 of operation of the SER system 20 of FIGS. 1 and 2. The method 100 commences with the initialization of the SER system 20 (110). Once the SER system 20 is initialized, the SER system is calibrated (120). After calibration, sanitization of one or more surfaces is performed (130). During and/or after the performance of the sanitization, feedback is collected regarding the dose of SER, the level of use of the area to be sanitized of which the surface(s) form(s) a part of, etc. (140).

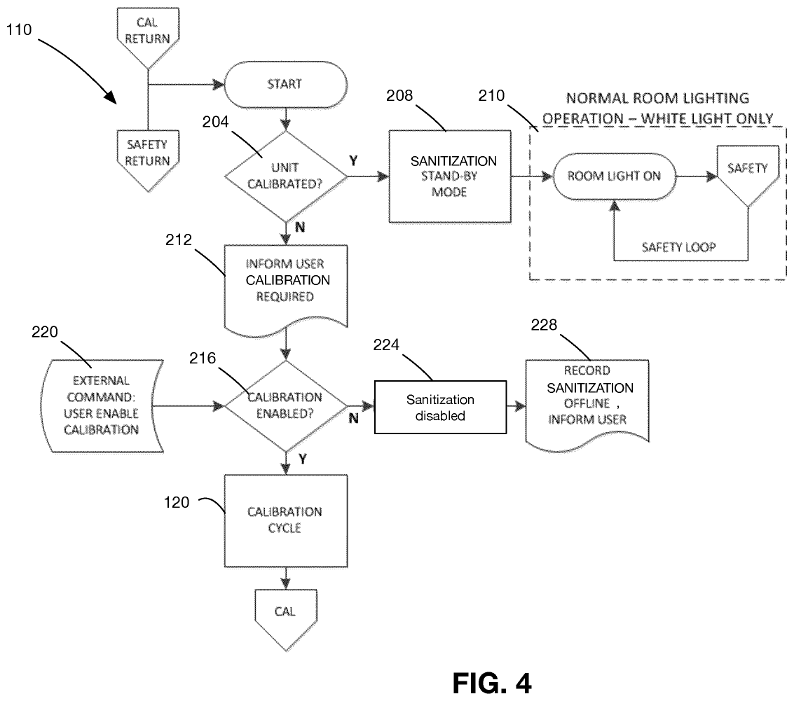

The process of initialization at 110 is shown in greater detail in FIG. 4. Initialization occurs when the SER system is powered up or as otherwise specified below. During initialization, the control module 28 checks if the SER system has been calibrated (204). If it is determined that the SER system 20 has been calibrated at 204, the SER system 20 enters sanitization stand-by mode (208). In sanitization stand-by mode, the SER system 20 enters a safety operation loop in which the control module 28 directs the illumination module 24 to illuminate the area with standard white light. Where the illumination module 24 does not include a standard white light, the control module 28 can interface with external lighting systems to illuminate the area with standard white light (210). The standard white light notifies people that sanitization cycle is not being performed now or in the near future.

If, instead, it is determined that the SER system 20 has not yet been calibrated at 204, a message is provided to the user that calibration is required (212). Users can be notified that calibration is required via a message sent from the control module 28 via a network, via a message displayed on the control module 28, or any other suitable manner for notifying users that calibration is required. It is then determined if calibration is enabled for the SER system 20 (216). Calibration can be enabled by a user (220) when calibration is not yet enabled for the SER system 20 via a button on the control module 28, via an external application that interfaces with the control module 28, or via any other suitable means. If it is determined at 216 that calibration is not enabled, sanitization is disabled (224), and the user is informed that sanitization is offline (228). Users can be notified that sanitization is disabled via a message sent from the control module 28 via a network, via a message displayed on the control module 28, or any other suitable manner for notifying a user that sanitization is disabled. If, instead, it is determined that calibration is enabled at 216, the control module 28 commences calibration 120.

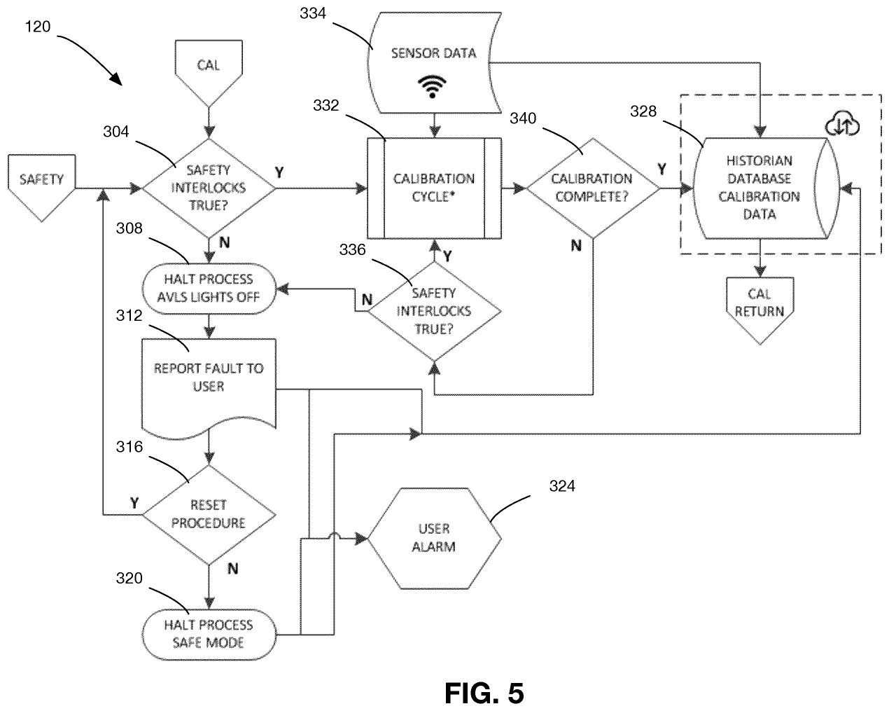

FIG. 5 shows the process of calibration 120 in greater detail. During calibration, the control module 28 performs a calibration on the output of the illumination module 24 to ensure that the appropriate dose of sanitization light is delivered to the surface(s), room(s), or space(s). An appropriate intensity, duration, frequency, and/or SER wavelength of the sanitization cycle is determined and registered to allow off-line use of the calibration parameters for ongoing sanitization cycles. In one embodiment, the calibration cycle may be real-time, informing and causing adjustments to be made to the operation of the illumination module 24 as it operates through its sanitization cycle, or it may be a periodic calibration with key parameters uploaded and stored in the database 68 to allow repeated operation using the stored calibration data.

The process of calibration 120 commences with a determination of whether a set of safety interlocks are true (304). Safety interlocks are a set of one or more conditions/rules that are to be met to enable generation of SER by the illumination module 24. These safety interlocks are stored in the database 68 of the control module 28, but can be stored in any suitable location that is accessible by the control module 28. Any suitable format for storage of the safety interlocks can be employed, and examples include XML and JSON. Examples of safety interlocks include the absence of movement in the area to be sanitized, a closure and/or a lock state of doors or ports providing access to the area to be sanitized, the deactivation of standard visible illumination in the area to be sanitized, etc. If it is determined at 304 that one or more of the safety interlocks is not satisfied, the calibration process is halted (308), and a fault is reported to users (312). Users can be notified of the fault via any suitable manner, such as a message sent from the control module 28 via a network, a message displayed on the control module 28, etc. It is then determined if the calibration procedure is to be terminated or await the satisfaction of the safety locks (316). The SER system can be configured in either manner. If the calibration procedure is to await the satisfaction of the safety interlocks, the process returns to 304 until the safety interlocks are satisfied. If, instead, it is determined at 316 that the calibration procedure is to be halted, the process is halted (320), after which users are alerted (324). Users can be alerted of the termination of the calibration process via a message sent from the control module 28 via a network, via a message displayed on the control module 28, or any other suitable manner for notifying users. Additionally, any calibration data collected including metadata such as the time the safety interlocks were satisfied or unsatisfied, etc. are logged in the database 68 (328). The calibration data is then communicated by the control module 28 to a remote server to enable remote monitoring and management of the SER system 20.

If it is determined at 304 that the safety interlocks are satisfied, calibration commences (332). During calibration, the illumination module 24 is operated and the output is measured by the dosimeter 32 (334). Numerous factors can lead to the need to recalibrate the SER system 20. One cause can be the degradation of the illumination module 24. Another example can be movement of the illumination module 24 relative to the surface(s) to be sanitized. Operation of the illumination module can be adjusted during calibration to determine the relationship with the output and determined the inputs required to provide a desired level of SER output. Calibration continues while monitoring the satisfaction of the safety interlocks (336) until it is determined that it is complete (340). If, during calibration, it is determined that one or more of the safety interlocks is not satisfied at 336, the calibration is halted at 308. Upon the determination of the completion of the calibration at 340, the calibration data collected as well as metadata such as the time of the calibration is logged in the database 68 at 328.

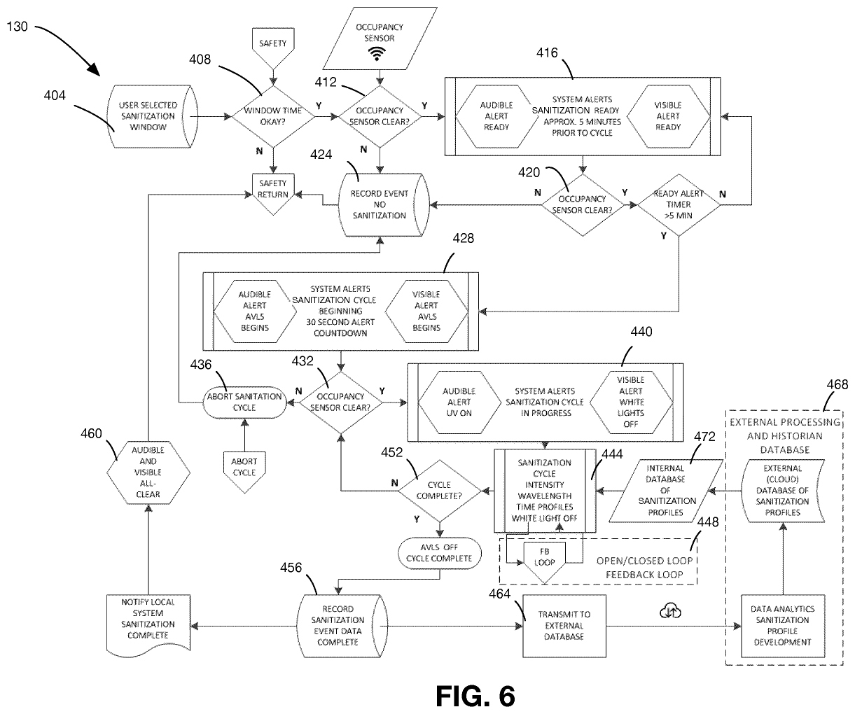

During the process of sanitization 130 illustrated in greater detail in FIG. 6, the illumination module 24 is operated by the control module 28 for a duration and at an intensity, as set during the calibration loop, to deliver a desired dose of sanitization light to the surface or surfaces in an area, space, or another desired environment. The sanitization step includes one or more safety interlocks to ensure that operation avoids undesired exposure to the SER. In one embodiment, for example, an occupancy sensor is used to ensure that the area being sanitized is unoccupied. In another embodiment motion sensors may be used to ensure that there is no motion within a space being sanitized, and upon detection of any motion, sanitization will immediately be halted.

In another embodiment, the calibration loop is essentially performed prior to each sanitization cycle, and real-time closed loop feedback is used in the operation, dynamically controlling the illumination module 24 intensity and duration to effect the desired dose of light for sanitization. In some embodiments, the operation of the SER system 20 includes user alerts of a pending, in process, successful, or halted sanitization cycle. Alerts can, for example, comprise visible warnings or indicators, which may be lights, signals, signs, or other indicators, that flash, illuminate, change intensity, or use other means to indicate a cycle is about to start, a cycle is ongoing, a cycle has faulted, a cycle has been halted, or for other reasons. Alerts may also include audible signals that sound or indicate or use other audible means to indicate a cycle is about to start, a cycle is ongoing, a cycle has faulted, a cycle has been halted, or for other reasons.

In another embodiment, the SER system 20 can communicate that a sanitization cycle is about to start, a sanitization cycle is ongoing, a sanitization cycle has faulted, a sanitization cycle has been halted, or for other reasons, through use of electronic messaging means, including email, Bluetooth, Wi-Fi, or other wireless communication means, or through other means or methods.

The sanitization process 130 commences with the occurrence of a user-selected sanitization time window (404). A user or operator can input a time or time window during which the sanitization cycle is to be run. Entry of the time or time window can be performed locally via buttons of the I/O interface 52 of the control module 28, remotely via an application, web interface, etc. This can include locking out certain times, selecting certain times, or other means to determine a window of operation time for the SER system 20.

Upon reaching that time or time window, it is determined if the time or time window specified by the user is ok (408). If it is determined that the time or time window is acceptable at 408, it is determined at that time or in that time window if an occupancy/motion sensor indicates that the area to be sanitized is clear (412). The occupancy/motion sensor can detect the presence of a person in the area to be sanitized via any suitable means. If movement is detected by the occupancy/motion sensor, the event is registered in the database 68 and the sanitization cycle ends. If, instead, no movement is detected in the area by the occupancy/motion sensor, the SER system 20 alerts persons in the area to be sanitized that a sanitization cycle is about to be performed for a period of time before the sanitization cycle is to be carried out (416). In particular, the control module 28 generates an audible alert via the speaker 53 and directs the illumination module 24 to alternatingly turn on and extinguish the standard light provided to the area. Where the illumination module 24 is not configured to provide standard lighting, the control module 28 can be configured to interface with the standard lighting system for the area to alert people that a sanitization cycle is scheduled to start.

If the occupancy sensor detects occupancy of the area via movement or otherwise at 420, the event that no sanitization is being performed is recorded in the database 68 (424). If, instead, the occupancy sensor does not detect occupancy of the area, the control module 28 alerts people in the area that the sanitization cycle is imminent 30 seconds before the sanitization cycle is scheduled to start (428). In particular, the control module 28 plays a different, more distinct audio alert and more frequently alternatingly turns on and extinguishes the standard light provided to the area.

If the occupancy sensor detects occupancy of the area at 432, the sanitization cycle is aborted at 436 and the event that no sanitization is being performed is recorded in the database 68 at 424. If, instead, the occupancy sensor does not detect occupancy of the area, at 432, the control module 28 generates an alert that a sanitization cycle is in progress (440). In one embodiment, the control module 28 plays a voice track stating that SER is being employed and turns off the standard white lights. Simultaneously, the control module 28 controls the illumination module 24 to provide SER lighting of an intensity, duration, and wavelength(s) determined during calibration (444). The control module 28 can also employ an open or closed feedback loop to adjust the dose of SER during the sanitization cycle (448). The occupancy sensor is continually monitored during the sanitization cycle to ensure that it is halted if occupancy is detected.

Once it is determined that the sanitization cycle is complete at 452, the completion of the sanitization cycle and the associated metadata, including the time of start and end, the intensity and wavelength(s) of the SER lighting, etc., are logged in the database 68 (456). Simultaneously, the control module 28 generates an alert that the sanitization cycle has ended (460). In particular, the control module 28 can direct the illumination module 24 to display a pattern of alternating illumination and extinguishing of standard white light that differs from the alternating pattern of standard white light generated prior to a sanitization cycle. In addition, the control module 28 transmits the logged data to an external database (464). The external database managed by the server 39 can store sanitization profiles, and calibration data from multiple SER systems 20. The server 39 can use data analytics to develop sanitization profiles (468) and push the sanitization profiles to the control modules 28 for modifying the SER dose regimens provided by the SER systems 20 (472).

During the sanitization cycle, the control module 28 can record occupancy data, faults, halts, successful cycles, or other data that is desired. This data may be used for analytics, to for example, adjust the duration, frequency, intensity, or wavelength of the SER light, or for other reasons.

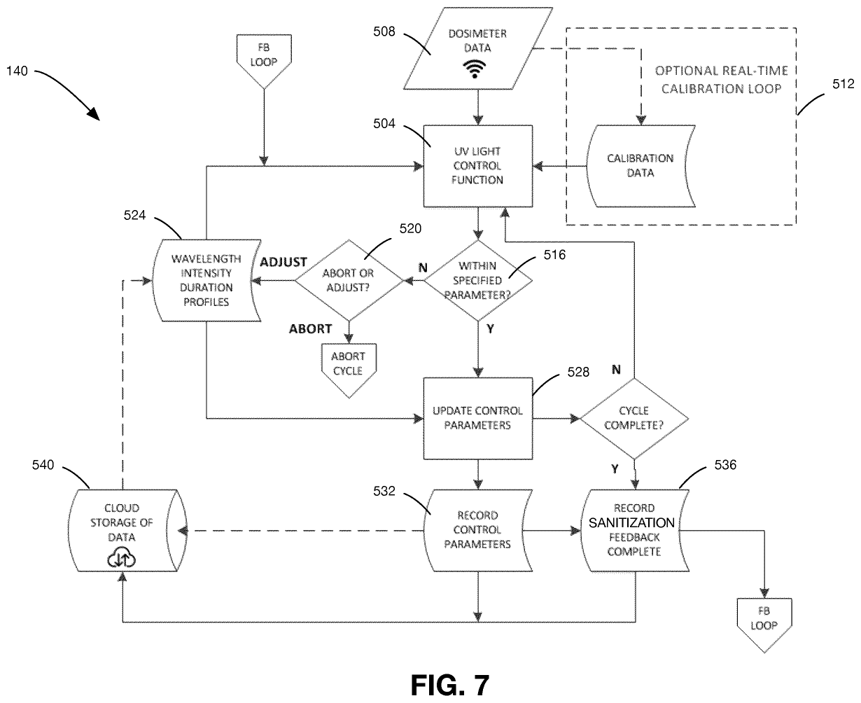

FIG. 7 illustrates the feedback process 140 in greater detail. The feedback step is normally embedded within one or more of the other processes but may comprise a separate process or cycle that supports the other processes. The feedback process includes means to collect and determine the wavelength, time, duration, and other operational factors to achieve the desired sanitization. The feedback step may be ongoing and real-time, continuously altering the operation of the SER light in response to data from sensors, or it may be one-time, providing a means to update various control parameters that are stored and used for illumination module 24 operation.

During the feedback cycle, the illumination module 24 is operated (504). Dosimeter data can be collected and analyzed (508), and this dosimeter data is used as calibration data to calibrate the operation of the illumination module 24 by the control module 28 (512). It is determined if the outputted SER light is within specified parameters (516). If it is determined that the outputted light is not within the specified parameters at 516, it is determined if operation of the illumination module 24 is to be aborted or adjusted (520). If operation of the illumination module 24 is to be adjusted, the wavelength(s), intensity, and duration profiles used to operate the illumination module 24 are adjusted (524). The control parameters are updated (528) and recorded (532), and it is recorded that the sanitization feedback is complete (536). The control parameters are communicated to the server 43 (540).

The illumination modules 24 of the SER system 20, and the SER system 20 as a whole, can be configured to be directly fit into existing luminaires and lighting fixtures in some scenarios on transit rolling stock, on platforms, inside shelters, inside elevators, on touch pads or operating panels, or elsewhere, taking the place of existing light bulbs and tubes. In one embodiment, the illumination module 24 can have the dimensions and electrical connections consistent with replacement of a standard T5 fluorescent tube-type bulb. In another embodiment, the illumination module 24 can be designed as a direct replacement for existing medium-based light bulbs (E26/27 base). In another embodiment, the illuminator module 24 can fit within GU10 recessed lighting fixtures. The illumination module 24 may also de designed to fit within other luminaires and other fixtures using standard or non-standard light-bulb packages. The illumination module 24 can be powered by 12 VDC, 24 VDC, other DC voltage as may be used on subways, commuter trains, buses, or other conveyances, or may operate using the AC mains as supplied by the power authority or onsite generation. The packaging of the light-emitting elements can be made to include the other components of the SER system 20.

The retrofit illumination module 24 may include connectivity to external controls, including mobile device applications, IoT devices, or other control or data collection devices.

The illumination module 24 can be designed to meet emerging standards for LED lights, including, but not limited to, flat panels, integrated LED recessed lighting fixtures, integrated LED surface mounted lighting fixtures, LED or standard lighting wall sconces, LED HID or other high bay lights, or other room and space lighting fixtures.

The new design illumination module 24 can include connectivity to external controls, including mobile device applications, IoT devices, or other control or data collection devices.

The interconnected set of hardware and software of the SER system can achieve greater assurances of sanitization in various deployments. Exemplary applications include transit spaces such as the interior of vehicles or subway platforms, within a “BOPIS” (buy online, pick-up in store) locker or other automated or manual product dispensers and vending machines, on conveyors such as those used in shopping centres and for luggage handling, on escalator handrails, in elevators, etc. Further, the SER system can be integrated with any of a variety of systems that may also include clothes dryers, food handling systems, agricultural grow systems, and a myriad of other applications.

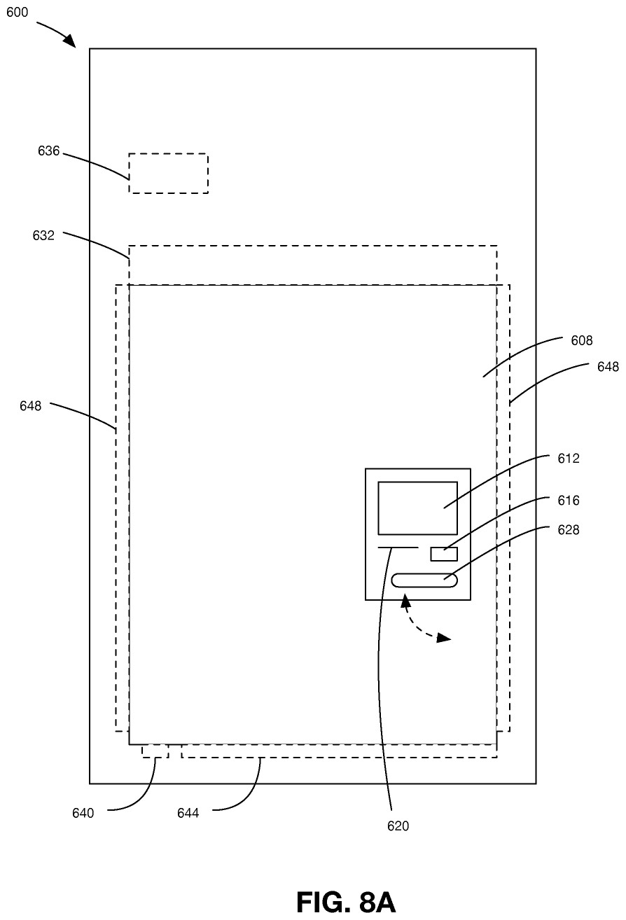

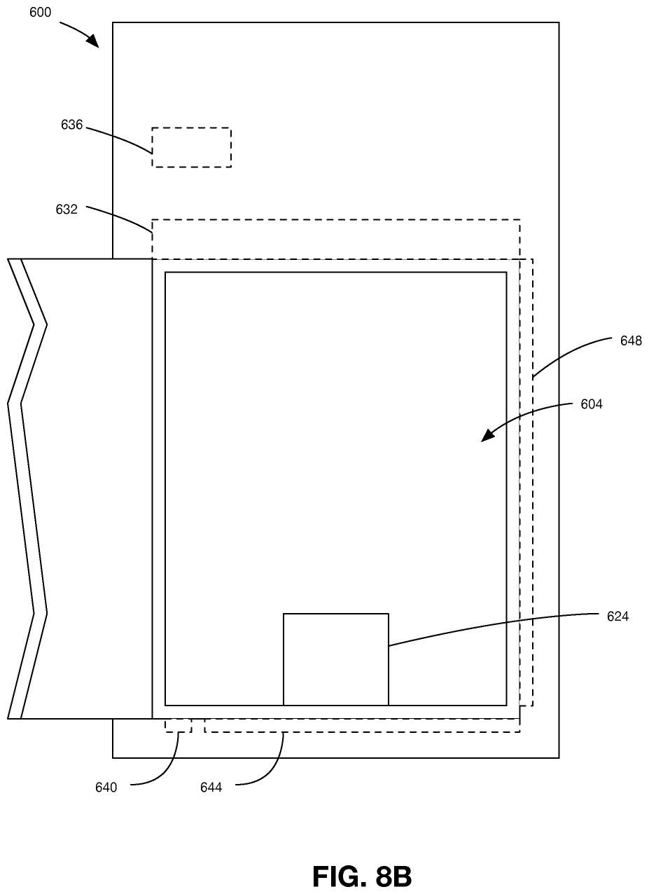

FIGS. 8A and 8B show a BOPIS locker 600 in accordance with an embodiment. It will be appreciated that similar principles can be applied to other types of lockers, vending machines, etc. The BOPIS locker 600 provides effective automatic sanitization of an interior space 604 thereof to provide greater assurance to customers that steps have been taken to sanitize the exterior surfaces of any package placed within the interior space 604 of the BOPIS locker 600, as well as the interior space 604 itself.

The BOPIS locker 600 has a door 608 that can be closed to inhibit access to the interior space 604, as shown in FIG. 8A, or open to provide access to the interior space 604, as shown in FIG. 8B. Located on the door 608 is an access control system that includes a touchscreen 612 to present information and/or alerts to a user and enable the user to make selections or enter information. In addition, the access control system includes a near-field communications (NFC) module 616 to enable communication between the BOPIS locker 600 and an NFC-enabled mobile device, credit or debit card, fob, etc. A card slot 620 enables payment to be received, if required. The access control system enables locking and unlocking of the door 608 to restrict or grant access to the interior space 604 and any items 624 stored therein. The door 608 can be opened or closed, once unlocked, via a handle 628.

An SER system is integrated into the BOPIS locker 600, and includes an illumination module 632 that is installed on an upper surface of the interior space 604, and is controlled by a control module 636. The illumination module can be the same as the illumination module 24 of FIG. 1 as described above. The control module 636 is connected to the illumination module 632 to control operation thereof. In addition, the control module can be connected to the access control system of the BOPIS locker 600 to detect when the door 608 is closed and locked, and when the door is unlocked. One of the safety interlocks employed by the SER system is the closed, locked state of the door 608. Preferably, the SER system is integrated with the access control system of the BOPIS locker 600 to disable unlocking of the door 608 while a calibration, sanitization, etc. is being performed. As a result, the SER system is able to determine when provisioning of SER lighting to the interior space 604 is safe.

In its position, the illumination module 632 is able to illuminate the top surfaces of an item 624 placed in the interior space 604 for customer collection. An additional bottom illuminator module 644 may be positioned on or below the floor of the interior space 604 below the item to effect sanitization of a bottom surface of the item 624. The additional bottom illumination module 644 can include a guided wave structure, a plate of UV transmissive material, etc., and can cover a portion, the majority, or the entirety of the bottom of the interior space 604 to guide and distribute SER light that is coupled into it onto the bottom surface of the item 624. This guided wave structure may have integral SER light sources as part of it or may guide light from the overhead illumination module 632 onto the bottom surface. Such guided wave structure materials include, but are not limited to, fused silica, crystalline silica (quartz), calcium fluoride (CaF2), lithium fluoride (LiF), magnesium fluoride (MgF2), artificial diamond, and borate crystals. Similarly, additional side illumination modules 648 can be positioned along the walls of the interior space 604 to effect sanitization of side surfaces of the item 624. The side illumination modules 648 can be constructed in a similar manner to the overhead illumination module 632 and/or the bottom illumination module 644. In this manner, all surfaces of the item 624 can be sanitized.

The control module 636 can present information regarding the sanitization of the interior space 604 and item 624. For example, a green light can be used to illuminate the interior space 604 or a mark/stamp can be placed on the item 624 itself upon the completion of a sanitization cycle. Conversely, also included is a means to indicate that sanitization has not occurred or was interrupted or delayed, which could include a visible indicator such as illuminating the interior with red light. In addition, the control module 636 can additionally or alternatively present information regarding the sanitization state of the interior space 604 and, thus, the item 624, using the touch screen 612.

The control module 636 can include interlocks with the access control system of the BOPIS locker 600, such that if the door 608 is not locked, the SER sanitization cycle cannot commence and if the door 608 unlocks during a sanitization cycle, the cycle is immediately stopped. Additionally, there may be included a means to indicate to the customer externally to the BOPIS locker 600 when the door 608 is closed that a sanitization cycle is in progress, and the customer may optionally wait for the cycle to complete or may interrupt the cycle and take the item 624 without a completed sanitization cycle.

In one present embodiment, the operation of a sanitization cycle for the BOPIS locker 600 is as follows. The item 624 to be picked up by the customer is placed in the interior space 604 of the BOPIS locker 600 by staff, and the BOPIS locker 600 closed. When the locker door 608 has been closed for an interval that is pre-determined, the SER sanitization light of the illumination module 632 will operate, covering the interior space 604 of the BOPIS locker 600 uniformly in SER light, and sanitizing the exterior surfaces of the item 624. The SER system will record the successful sanitization event and prepare the visual indicator for display to the customer on the touchscreen 612 when the item 624 is retrieved. The customer uses the access control system to retrieve the item 624, identifying themselves using the touchscreen 612, the NFC module 616, and/or the card slot 620 to unlock the locker door 608. Upon receipt of the correct unlock information, the locker interior space 604 will illuminate green to indicate that the item 624 has undergone a successful sanitization cycle, and the door 608 will unlock.

If the sanitization cycle fails, the interior space 604 will illuminate red to indicate that the sanitization cycle was not successful, providing a visual indicator to the customer. The customer will be given the opportunity to interrupt an in-process sanitization cycle to enable early collection of the item 624, but as with a failed event, the interior space 604 of the BOPIS locker 600 will illuminate red to indicate an incomplete sanitization cycle. Upon closing the door 608 after retrieving the contents from the interior space 604 of the BOPIS locker 600, the sanitization cycle will again initiate, sanitizing the BOPIS locker 600 in preparation for the next loading event by staff.

Closed loop feedback provides a means for a remote dosimeter to record the real-time dose of UV that is delivered to the dose sensor and communicate back to the illumination modules 632, 644, 648 to adjust the light delivered to meet the required sanitization specification. This system can self-adjust for desired dose, compensating for any changes in the SER illumination modules' efficiency (dust on the illumination module 632 for example) and provides the greatest assurance of sanitization, recording the time, duration, and dose for each sanitization cycle. This data can be collected and stored locally by the control module 636 with which the illumination modules 632, 644, 648 is in communication or broadcast directly to the cloud for subsequent data analytics. The data ca be used by organizations and property managers to correlate to sick days for example against baseline.

The calibrated open loop operation uses the same or a similar dosimeter to record delivered UV sanitization during installation and set-up of the illumination modules 632, 644, 648, communicating received sensor information back to the illuminator modules 632, 644, 648 for subsequent open loop operation. The illuminator modules 632, 644, 648 then enter a “repeat mode”, delivering the timed intensity dose determined during the set-up on each sanitization event. There is no real-time monitoring, and the system requires periodic re- calibration to ensure it is operating within desired parameters. Continuous monitoring of safety interlocks ensure that the sanitization cycle is immediately stopped should the locker door 608 be opened by any means during a sanitization cycle, and the failed sanitization event logged for future reference. The completed sanitization cycle is logged, and the system re-enters “standby mode”, with an appropriate record of the event.

In some configurations, the illuminator modules 632, 644, 648 can include wireless technology allowing connectivity as an IoT device (internet of things), or through dedicated wireless communication using Wi-Fi, Bluetooth, Zigbee, or another suitable wireless protocol. This connectivity provides a means for the adjustment, in real-time if desired, of the sanitization illumination wavelength, which in some embodiments includes, for example, the entire range of UVC at 200 nm through to UVA at up to 400 nm, or other desired wavelength, and the intensity of illumination. The control loop may be through a calibrated external dosimeter that will measure the delivered dose of SER light at a desired surface or height within a room, or through a real-time feedback control using a connected dosimeter to communicate in real-time with the illuminator module through the sanitization cycle, or the illuminator module may be calibrated off-line and operated in an open-loop mode.

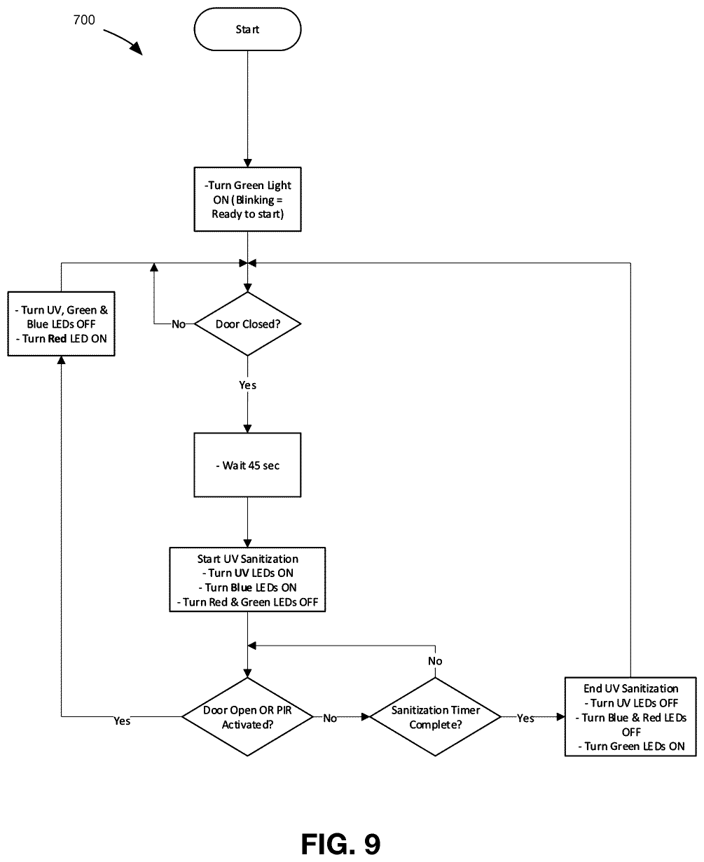

FIG. 9 outlines a generic control cycle 700 for the BOPIS locker 600 or another vending/dispensing machine. The general steps and requirements to achieve the sanitization are as follows:
a means to detect that the BOPIS locker 600 is closed, and that users do not have access to the interior space 604;
a means to trigger the start of a sanitization cycle, which may include for example, include the following elements:
   a delay of after the door 608 has closed before beginning the sanitization cycle;
   a trigger to the start of the sanitization cycle, causing the SER light to illuminate the interior space 604;
   a means to detect an interruption in the sanitization cycle, for example through a premature opening of the door 608, and a means to immediately halt the cycle;
   a means to time the operation of the SER light to limit the operation to a specific time;
   a means to visually indicate with ancillary lighting the ongoing operation of the SER sanitization light;
   a means to immediately halt the sanitization cycle if the door 608 opens at any point during the sanitization cycle, and a means to indicate to users that a fault has occurred;
   a means to indicate a successful SER cycle and a means to indicate a successful sanitization cycle to the user;
a means to reset the system to be ready for the next sanitization cycle upon opening and closing of the locker door 608

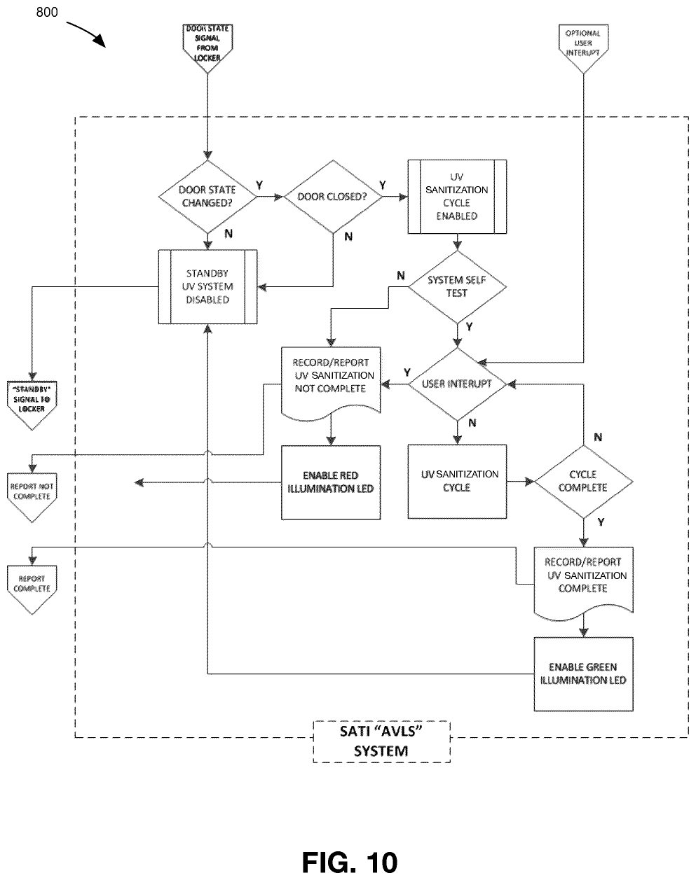

FIG. 10 outlines an alternative control loop 800 for sanitization in the BOPIS locker 600 or a similar device, such as a vending machine.

The SER system is applicable to sanitizing surfaces that are moving, such as conveyors and moving handrails.

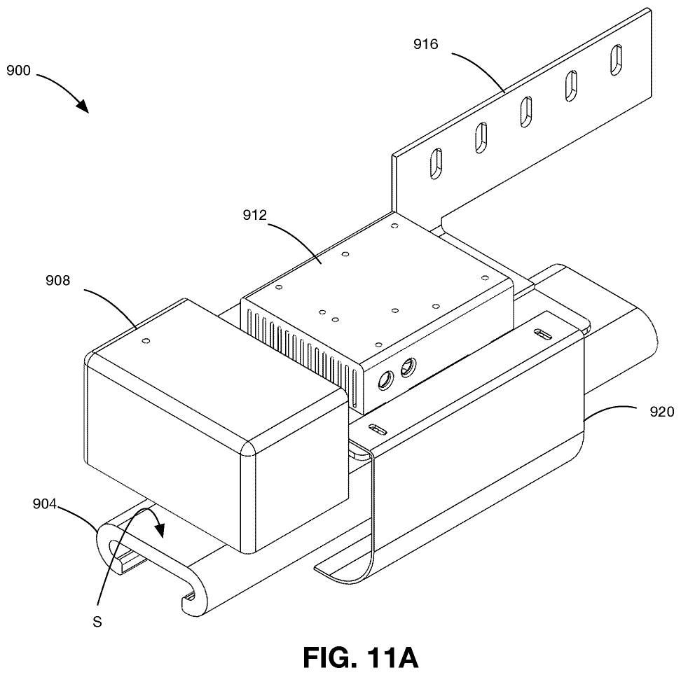

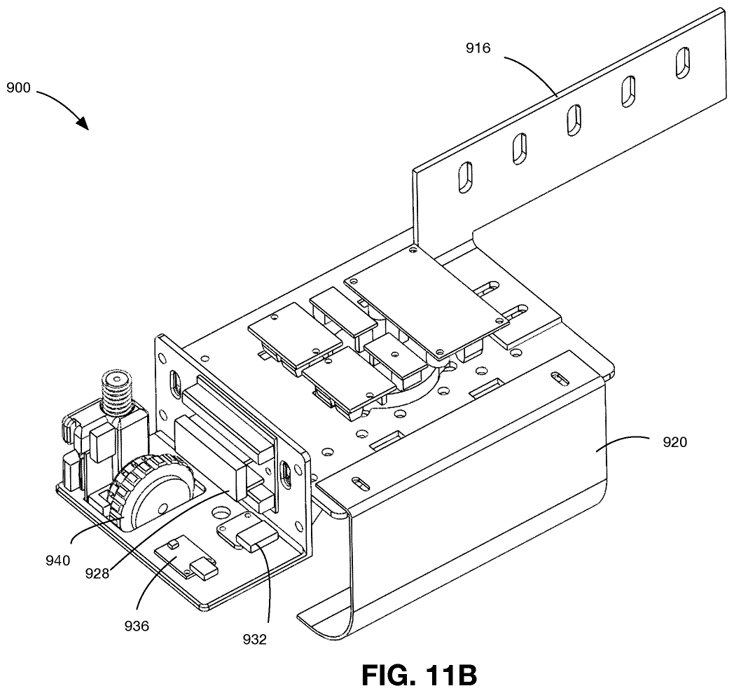

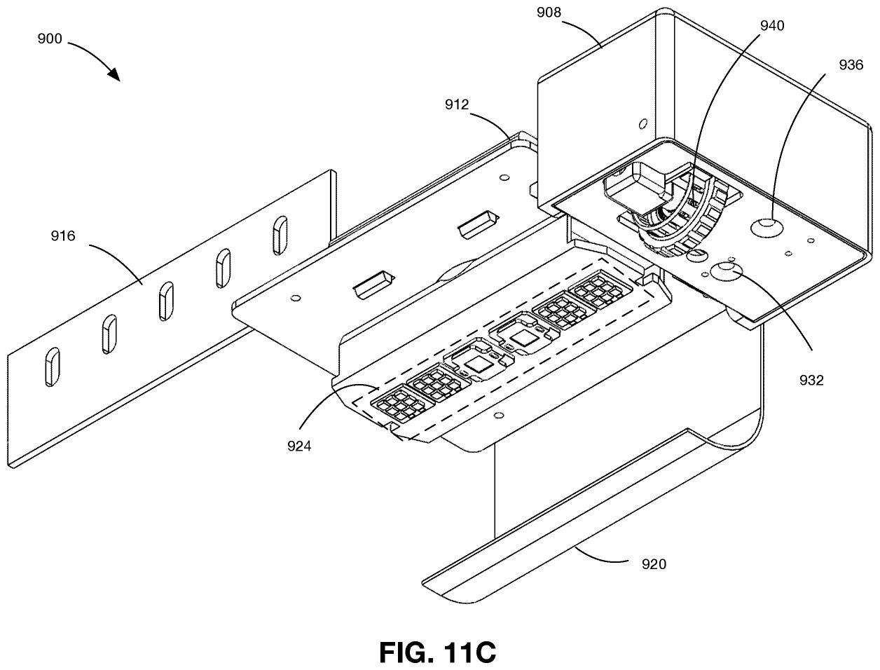

FIGS. 11A to 11C illustrate an SER system 900 for sanitizing a surface S of a moving handrail 904 of an escalator in accordance with yet a further embodiment. The SER system 900 can verify that that the surface S is properly sanitized to a specified level. The SER system 900 has two main functions: SER light sanitization of the surface S of the escalator handrail 904, and verification of cleanliness of the handrail 904. Preferably, the SER system 900 is to be installed in a hidden area within the handrail service bay or in the area below a conveyor system and will not be in direct contact with users to facilitate the containment of the SER radiation emitted by the SER system 900. In other embodiments of the SER system, it can also be used to sanitize and verify cleanliness of the surface of handrails used for moving walkways, and any conceivable conveyors or similar systems.

The SER system 900 includes a control unit 908 that contains the logic controller and sensors, and a power module 912 that houses the power supplies and LEDs in for the system. It will be appreciated that the various components of these two modules can be combined into a single module and placed on a single PCB controller that is designed to carry out these tasks, or split into a number of additional modules. For simplicity, these two modules are described below individually. A mounting bracket 916 enables mounting of the SER system 900 to structure of the escalator or another suitable location to position the SER system 900 adjacent to the handrail 904. A light reflector 920 inhibits the escape of SER light from the SER system 900, shielding persons within the vicinity of the SER system 900 during calibration or sanitization.

The power module 912 includes LEDs 924 or other light emitters for emitting SER light to carry out the sanitization. This SER light serves to sanitize the surface S of the handrail 904. Typically, the power delivered by the SER light is in the 10-100 W range, and wavelengths may include any light that effects sanitization functions, but is typically 270-280 nm (UVC), and 380-385 nm (UVA). These are the preferred power and wavelength ranges but can be adjusted as needed depending on the power requirements or availability of light sources to include wavelengths throughout the entire UV frequency spectrum (10-400 nm) or even beyond.

Coolers to dissipate the heat generated by the LEDs 924 or other illumination elements are included (optional depending on total heat output and ventilation conditions in service bay).

A power supply is used to convert the input power available, typically 110 or 220 VAC at 50 or 60 Hz, to an appropriate level of DC power to be used by the LEDs or other light source available.

The control unit 908 includes a control module 928 that executes the method(s) of sanitizing the surface S of the handrail 904, and uses the signals from the sensors described below in the control unit 908 to regulate power to the power module 912 and the light sources within it. The sensors include a cleanliness sensor 932 that verifies the cleanliness of the handrail 904 by sensing the amount of visible light reflected from the handrail 904, it can determine if the handrail 904 is clean or if it has foreign material deposited on it that needs to be manually removed.

A dosimeter 936 verifies that the required level of light radiation has reached the handrail 904 to provide adequate sanitization. It measures light, typically UV radiation, that is either reflected or scattered by the handrail 904 to confirm that the appropriate level of UV radiation has been delivered. Alternatively, the dosimeter 936 can also be mounted on the underside of the handrail 904 or tip of the light reflector 920 to ensure enough UV radiation is delivered to areas not easily reached.

A use sensor 940 confirms that the handrail is moving and its relative speed to adjust power level of the LEDs 924 accordingly. The use sensor 940 includes a wheel that impinges upon the handrail 904 to detect movement of the handrail 904. This function can be achieved by using several different sensor types; in some embodiments, this sensor can be a tachometer, an encoder, a doppler effect sensor, radar, ultrasonic sensor, etc.

Once motion of the handrail 904 is verified, the control module 928 uses the speed reading to adjust the UV radiation to the appropriate level proportional to the relative speed of the handrail 904 (e.g., if the handrail 904 is travelling at 50% of its speed setpoint, the control module 928 will send 50% of the output power to the LEDs 924).

The control module 928 uses the data from the dosimeter 936 to verify that the UV radiation delivered is within the expected levels for sanitization. If the radiation is adequate, it will report that the handrail 904 is sanitized and return to the start of the control cycle. However, if the dosimeter 936 does not report the required level of UV radiation, the control module 928 can adjust the power intensity of the LEDs 924, report that sanitization is “in progress” and return to the start of the cycle. Sanitization status will then be verified in the next iteration of the cycle.

Like the previous control loop, handrail cleanliness will only be executed once the handrail is verified to be in motion, otherwise cleanliness will be subject to the area of the handrail being measured only. The control module 928 uses data from the cleanliness sensor 932 to relate it to a predetermined status of cleanliness of the handrail 904. As the handrail 904 gets dirty, it will reflect less light back to the cleanliness sensor 932 and once the reflectivity drops below a specified threshold, the control module 928 determines that the handrail 904 is dirty and notifies an operator that manually cleaning of the handrail 904 is required.

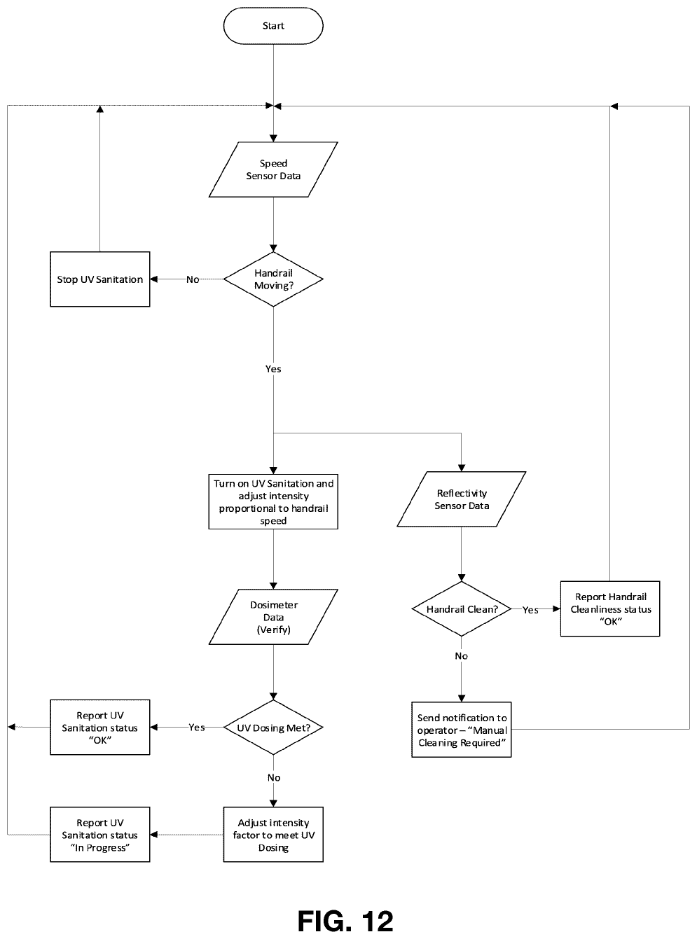

FIG. 12 shows the logic 1000 used to verify that the surface S of the handrail 904 has been satisfactorily cleaned using SER light from the LEDs 924. The overall process begins with the verification that that handrail 904 is moving. If the handrail 904 is stopped, then UV radiation is also stopped to prevent damage to the handrail 904 and the control module 928 continues to monitor movement of the handrail 904 until it starts moving. Once the handrail 904 is in motion, the control module 928 tracks two separate processes independently, UV sanitization and handrail cleanliness verification, both of which cycle back to verifying handrail motion at the end of each iteration.

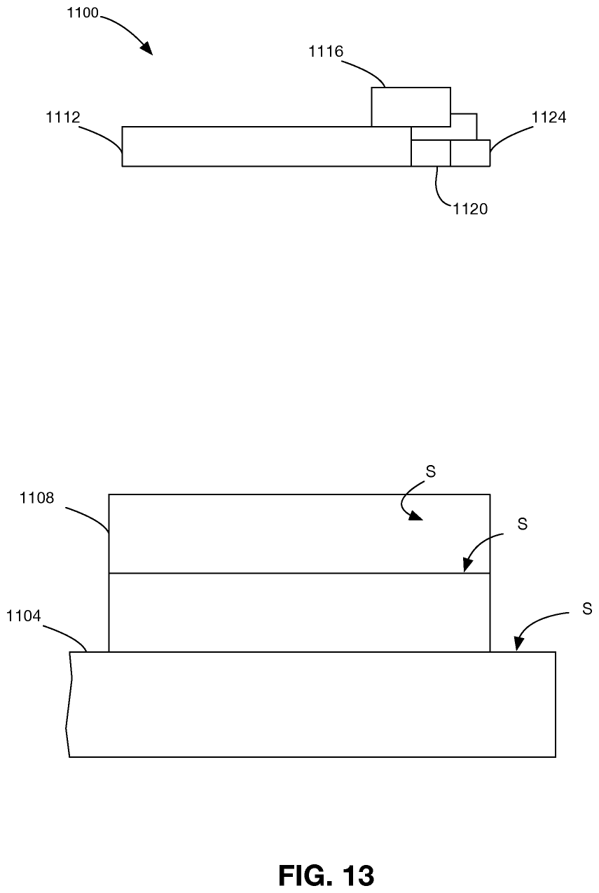

FIG. 13 shows an SER system 1100 in accordance with a further embodiment for use in a transit environment, such as a vehicle interior, seating on a platform, an office, a bus shelter, an elevator, or any other suitable public space. The particular transit environment illustrated is a subway car 1104 with seating 1108. The subway car 1104 and the seating 1108 have surfaces S that can come into contact with contaminants and, more particularly, pathogens. In one embodiment the SER system 1100 will operate on subway cars at the terminus stations; after all passengers have exited the car and before the subway car 1104 is returned to service the SER system 1100 will sanitize the interior surfaces S of the subway car 1104 and the seating 1108 by illuminating with UV light, with controlled and selected wavelengths, and including the option of dosimetry, proximity, and occupancy sensors.

The SER system 1100 includes an illumination module 1112 that is controlled by a control module 1116. The SER illumination module 1112 may be a replacement for an existing lighting system, providing the normal white light illumination, then switching to SER sanitization, or it may be separate from the existing lighting. The control module 1116 is also in communication with an occupancy/motion sensor 1120 and a dosimeter 1124.

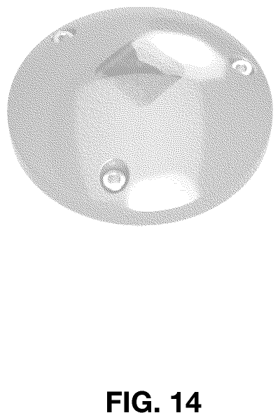

FIG. 14 shows a ceiling mounted sensor module 1132 that includes both the occupancy/motion sensor 1120 and the dosimeter 1124.

Returning again to FIG. 13, the SER system 1100 operates in the same manner as the office and public space system, with the only difference being inclusion of specific sanitization events that associate with the location of the rolling stock. For example, when a subway car 1104 (or light rail, bus, or other public transit conveyance in which it is positioned) reaches a terminus point, the vehicle is typically emptied, and the vehicle taken out of service or readied for a reverse trip. In either case, the vehicle doors will be closed, and the interior will be readied for SER sanitization.

The SER system 1100 uses the occupancy/motion sensor 1120 to ensure that the vehicle is empty prior to initiating any sanitization cycle. The occupancy/motion sensor 1120 may include proximity, movement, occupancy, or other sensors. Alternatively or additionally, there may be operational protocols that include in-person inspection.

Once the vehicle is determined to be empty, and thus safe to begin a sanitization cycle, the system begins the sanitization protocol. This protocol mirrors the cycle used in the office system, with provisions for all safety interlocks and operational modes. The sanitization cycle may also be initiated at other times in the operation of the conveyance. The system will include the same or similar audible, visible, haptic, or other indicators of the state of operation of the sanitization system, and the state of sanitization; for example, in one embodiment, the vehicle or platform illumination will change to amber after a pre-set or dynamically selected time/occupancy period elapses without sanitization to indicate that the system, conveyance, platform, room, or space require a sanitization cycle.

The SER system 1100 is designed with safety as the first priority, and the various control loops all integrate a safety control loop as part of their operation. The remaining control-loops comprise the initialization cycle, calibration cycle, sanitization cycle, and the feedback cycle. Each of these in accordance with a particular embodiment is described in some detail below with respect to FIGS. 4 to 7 above.

The SER system 1100 goes through an initialization cycle upon each power-up event, as shown in FIG. 4. This cycle comprises the various calibration and safety check required to ensure that the illumination module 1112 is in an appropriate “ready” state to undertake subsequent lighting and sanitization cycles. The cycle commences with the checking of the internal system for the calibration state—if the illumination module 1112 is within the allowed range of the calibration set point, it immediately moves into “sanitization stand-by mode”. This mode allows normal operation of the standard transit lighting in white-light mode and enters the safety loop to continuously ensure that the light is not operating incorrectly, and that sanitization cycles are only being undertaken with the allowed operational envelope. This operational envelope may include additional checks for safety to be undertaken by the transit staff, and/or locational checks such as ensuring that conveyances are offline (temporarily or for a shift), and that vehicles are empty through manual checks.

If the calibration state is not within the set point range, that is, the illumination module 1112 is out of calibration, there is the option for the system to provide immediate feedback to the user, and the illumination module 1112 waits for an external command to begin the calibration loop. This external command can originate from the local environment, or can be enabled using internet connectivity remotely, or can be initiated using other means.

If the calibration loop is not enabled within a pre-set time interval after the out-of-calibration error is detected, then the illumination module 1112 reverts to “transit light only mode”. In this mode, the illumination module 1112 only operates as a standard room light, delivering white light illumination only.

The event of “out of calibration error” is recorded, and the user informed of the failure. Additional records may result, either locally, through a wireless connection to a record keeping device, to the cloud for storage or other data manipulation, or other means. In some embodiments, a warning indicator on the illumination module, or transmitted from the unit to receiving unit, or transmitted to a wireless device such as a cellular phone, or to other devices using other means may be included.

If the calibration loop is enabled within the pre-set time interval after the out-of-calibration error is detected, then the illumination module 1112 will move into the “calibration cycle” as described below. Upon completion of the calibration cycle, the illumination module 1112 re-starts back at the beginning, going through the same safety and validation procedures.

The calibration cycle allows the illumination module 1112 to self-calibrate its output of SER light, to ensure that the correct dose of energy is delivered to surfaces and spaces within the vehicle, platform, station, shelter, or other transit space to effect the desired pathogen reduction (in this example, COVID-19). As with all control loops, the importance of safe operation is at the forefront, and checks of safety interlocks are continuously made throughout the cycle. Checks are also made to ensure that there are no occupants or passengers, and the cycle may include in some embodiments, specific manual intervention to initiate the calibration cycle.

The calibration cycle starts with a check of all safety interlocks. Safety interlocks include, occupancy sensors, proximity sensors, door sensors, power monitors, manual checks and protocols, location sensors, or other means to affect the safe operation of the sanitization UV light. The logic of the system delivers a “TRUE” reading when all the safety interlock sensors such as the occupancy/motion sensor 1120. included in the illumination module 1112 and connected externally to the unit using wireless or wired or other connection means, deliver a TRUE signal, that is, when all are within their safe operation set point range. If any one, or more than one, sensor is FALSE, then the safety interlocks will halt the process. This results in halting of the sanitization or calibration, including the termination of SER illumination. A report is generated to inform the user, and the report may be filed offline.

The illumination module 1112 enters a state looking for manual reset to restart the process—if reset command is received within the pre-set interval, then the process restarts at the beginning. If the safety interlocks are TRUE, then the illumination module 1112 will enter the calibration cycle, receiving data from the wireless or wire connected dosimeter 1124, and using this received reading to set the calibration of the SER light to be used for sanitization.

At any time if any of the safety interlocks enters a FALSE condition, then operation of the illumination module 1112 is immediately halted by the control module 1116, and it records and transmits the fault, and informs the user. Successful calibration data is stored locally and transmitted externally for further data analysis and quality assurance.

The sanitization cycle involves the operation of the SER light to affect the sanitization of the vehicle, platform, station, or other space on the transit system, using the calibration data, and may include closed loop feedback from the wireless or wired dosimeter 1124.

The feedback loop refers to the general feedback mechanisms for both closed and open loop operation of the illumination module 1112 and is shown in FIG. 7. Various control points, including abort and UV real-time or open loop features are incorporated to ensure that the illumination module 1112 affects the desired illumination profile. These can include specific wavelength, intensity, and duration profiles, either individually, or in combination, or other UV illumination protocols to affect the desired sanitization.

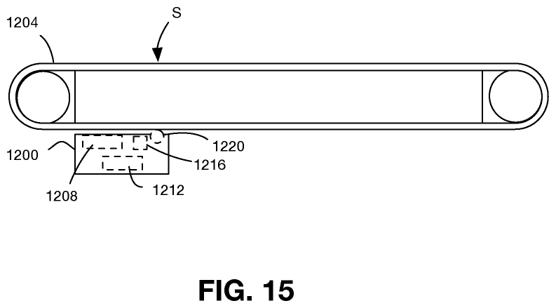

FIG. 15 shows an SER system 1200 for sanitizing a surface S of a conveyor belt 1204 in accordance with yet another embodiment, such as for conveying groceries at a cashier station. The SER system 1200 includes an illumination module 1208 that is configured to illuminate the surface S of the conveyor belt 1204 with SER. A control module 1212 controls operation of the illumination module 1208. A dosimeter 1216 is positioned to measure the SER light provided to the surface S of the conveyor belt 1204 by the illumination module 1208. A motion sensor 1220 includes a wheel that impinges on the surface S to detect motion of the conveyor belt 1204.

The control module 1212 controls operation of the illumination module 1208 much in the same manner as in the SER system of FIGS. 11A to 11C. The conveyor belt 1204 either operates at a set speed or does not move. The control module 1212 this motion or lack of motion is detected by the motion sensor 1220 and communicated to the control module 1212. When the conveyor belt 1204 is moving, the control module 1212 controls the illumination module 1208 to illuminate the surface S of the conveyor belt 1204 with SER light, and when the conveyor belt 1204 is not moving, the control module 1212 either doesn't illuminate the surface S or performs a calibration.

In other embodiments, such as luggage conveyor belts, assembly line conveyors, etc., the conveyor belts can operate at more than one speed. In such cases, the SER system can be configured to correspondingly increase the intensity of the SER light when the conveyor belt is moving more rapidly, or decrease the intensity of the SER light when the conveyor belt is moving less rapidly.

While the control module is shown as a single physical unit, it will be appreciated that the control module can include two or more physical units in communication with each other. Accordingly, while the embodiment shows the various components of the control module residing on the same physical device, those skilled in the art will appreciate that the components can reside on separate physical devices.

Although specific advantages have been enumerated above, various embodiments may include some, none, or all of the enumerated advantages.

Persons skilled in the art will appreciate that there are yet more alternative implementations and modifications possible, and that the above examples are only illustrations of one or more implementations. The scope, therefore, is only to be limited by the claims appended hereto.

What is claimed is:

1. A system for sanitizing at least one surface in an area to be sanitized, comprising:
at least one illumination module positioned to radiate the at least one surface with sanitizing electromagnetic radiation (SER);
a dosimeter positioned to measure a level of SER emitted by the at least one illumination module or received by the at least one surface; and
a control module in communication with the dosimeter to receive the level of SER emitted by the at least one illumination module or received by the at least one surface, determine an appropriate level of SER to be provided to the at least one surface at least partially based on the level of SER emitted by the at least one illumination module or received by the at least one surface, and control the at least one illumination module to radiate the at least one surface with the determined appropriate level of SER.

2. The system of claim 1, further comprising a use sensor positioned to determine a level of use of the area to be sanitized, and wherein the control system determines the appropriate level of SER to be provided to the at least one surface at least partially based on the level of use of the area to be sanitized.

3. The system of claim 2, wherein the use sensor is a motion sensor.

4. The system of claim 3, wherein the at least one surface includes a surface of a handrail.

5. The system of claim 3, wherein the at least one surface includes a surface of a conveyor belt.

6. The system of claim 1, wherein the control module includes storage storing a set of rules specifying conditions during which SER can be radiated by the at least one illumination module, and wherein the control module controls the at least one illumination module to only emit SER when the set of rules are satisfied.

7. The system of claim 6, wherein at least one of the set of rules specifies the absence of occupation or movement in the area to be sanitized.

8. The system of claim 6, wherein at least one of the set of rules specifies the closure or a lock state of a door or port providing access to the area to be sanitized.

9. The system of claim 6, wherein at least one of the set of rules specifies a deactivation of standard visible illumination in the area to be sanitized.

10. The system of claim 6, wherein the control module generates one of an audible alert and a visual alert when at least one of the set of rules is unsatisfied.

11. The system of claim 6, wherein the control module logs a failure event in a database in the storage when at least one of the set of rules is unsatisfied.

12. The system of claim 1, wherein the control module operates the illumination module to produce the appropriate level of SER according to at least one sanitization profile that specifies a duration, an intensity, and at least one wavelength of light.

13. The system of claim 12, wherein the control module performs a calibration to verify the appropriate level of SER by directing the at least one illumination module to generate a level of SER, measuring the intensity of SER using the dosimeter, and registering an adjustment for the at least one sanitization profile based on the measured intensity of SER.

14. The system of claim 13, wherein the calibration is performed during the provisioning of SER by the at least one illumination module to update the intensity in real-time.

15. The system of claim 12, wherein the control module is in communication with a server to receive one or more updated sanitization profiles from the server.

16. The system of claim 1, wherein the at least one illumination module is equipped to produce at least two wavelengths.

17. The system of claim 1, wherein the at least one illumination module is equipped to produce at least two wavelengths of SER.

18. The system of claim 1, further comprising a surface contamination sensor positioned to measure a level of surface contamination on the at least one surface, wherein the control module is in communication with the surface contamination sensor to receive the level of surface contamination and generate an alert when the level of surface contamination on the at least one surface surpasses a threshold.

19. The system of claim 1, wherein the control module provides at least one of an audible alert and a visual alert prior to controlling the at least one illumination module to radiate the at least one surface.

20. A system for sanitizing at least one surface of an area to be sanitized, comprising:
at least one illumination module positioned to radiate the at least one surface with sanitizing electromagnetic radiation (SER);
a use sensor positioned to determine a level of use of the area to be sanitized; and
a control system in communication with the use sensor to receive the level of use of the area to be sanitized, determine an appropriate level of SER to be provided to the at least one surface at least partially based on the level of use, and control the at least one illumination module to radiate the at least one surface with the determined appropriate level of SER.

21. The system of claim 20, wherein the at least one surface includes a moving surface, and wherein the use sensor detects movement of the moving surface and determines the appropriate level of SER to be provided to the at least one surface at least partially based on the movement of the moving surface.

22. The system of claim 21, wherein the use sensor detects a rate of the movement of the moving surface and determines the appropriate level of SER to be provided to the at least one surface at least partially based on the rate of movement of the moving surface.

23. A method for sanitizing at least one surface in an area to be sanitized, comprising:
measuring a level of sanitizing electromagnetic radiation (SER) emitted by at least one illumination module or received by the at least one surface;
determining an appropriate level of SER to be provided to the at least one surface at least partially based on the level of SER emitted by the at least one illumination module or received by the at least one surface; and
controlling the at least one illumination module to radiate the at least one surface with the determined appropriate level of SER.

24. The method of claim 23, further comprising:
determining, via a use sensor, a level of use of the area to be sanitized; and
determining, via a control module, the appropriate level of SER to be provided to the at least one surface at least partially based on the level of use of the area to be sanitized.

25. The method of claim 24, wherein the use sensor is a motion sensor.

26. The method of claim 25, wherein the at least one surface includes a surface of a handrail.

27. The method of claim 25, wherein the at least one surface includes a surface of a conveyor belt.

28. The method of claim 23, further comprising:
controlling, via a control module, the illumination module to only emit SER when a set of rules stored in storage are satisfied, the set of rules specifying conditions during which SER can be radiated by the at least one illumination module.

29. The method of claim 28, wherein at least one of the set of rules specifies the absence of occupation or movement in the area to be sanitized.

30. The method of claim 28, wherein at least one of the set of rules specifies the closure or a lock state of a door or port providing access to the area to be sanitized.

31. The method of claim 28, wherein at least one of the set of rules specifies a deactivation of standard visible illumination in the area to be sanitized.

32. The method of claim 28, further comprising generating, via the control module, one of an audible alert and a visual alert when at least one of the set of rules is unsatisfied.

33. The method of claim 28, further comprising logging, via the control module, a failure event in a database in the storage when at least one of the set of rules is unsatisfied.

34. The method of claim 23, further comprising controlling, via the control module, the at least one illumination module to produce the appropriate level of SER according to at least one sanitization profile that specifies a duration, an intensity, and at least one wavelength of light.

35. The method of claim 34, further comprising performing, via the control module, a calibration to verify the appropriate level of SER by directing the at least one illumination module to generate a level of SER, measuring the intensity of SER using the dosimeter, and registering an adjustment for the at least one sanitization profile based on the measured intensity of SER.

36. The method of claim 35, wherein the calibration is performed during the provisioning of SER by the at least one illumination module to update the intensity in real-time.

37. The method of claim 34, further comprising receiving, via the control module, one or more updated sanitization profiles from a server with which the control module is in communication.

38. The method of claim 24, wherein the at least one illumination module is equipped to produce at least two wavelengths.

39. The method of claim 23, wherein the at least one illumination module is equipped to produce at least two wavelengths of SER.

40. The method of claim 23, further comprising:
measuring, via a surface contamination sensor, a level of surface contamination on the at least one surface;
receiving, via the control module, the level of surface contamination from the surface contamination sensor; and
generating an alert when the level of surface contamination on the at least one surface surpasses a threshold.

41. The method of claim 23, further comprising providing, via the control module, at least one of an audible alert and a visual alert prior to controlling the at least one illumination module to radiate the at least one surface.

42. A method for sanitizing at least one surface in an area to be sanitized, comprising:
determining, via a use sensor, a level of use of the area to be sanitized;
determining, via a control module, an appropriate level of sanitizing electromagnetic radiation (SER) to be provided by at least one illumination module to the at least one surface at least partially based on the level of use; and
controlling the at least one illumination module to radiate the at least one surface with the determined appropriate level of SER.

43. The method of claim 42, wherein the at least one surface includes a moving surface, the method further comprising:
detecting, via the use sensor, movement of the moving surface; and
determining, via a control module, the appropriate level of SER to be provided to the at least one surface at least partially based on the movement of the moving surface.

44. The method of claim 42, further comprising:
detecting, via the use sensor, a rate of the movement of the moving surface; and
determining the appropriate level of SER to be provided to the at least one surface at least partially based on the rate of movement of the moving surface.
